(19) United States
(12) Patent Application Publication
(10) Pub. No.: US 2011/0129890 A1
(43) Pub. Date: Jun. 2, 2011

(54) METHOD OF TREATING SUBSTANCE CONTAINING LIGNOCELLULOSE OR CELLULOSE

(75) Inventor: Yukuo Katayama, Tokyo (JP)

(73) Assignee: K.E.M. CORPORATION, Tokyo (JP)

(21) Appl. No.: 13/056,189

(22) PCT Filed: Jul. 30, 2008
(86) PCT No.: PCT/JP2008/063678

§ 371 Date: Jan. 27, 2011

(51) Int. Cl. C12P 7/10; C13K 1/02

(52) U.S. Cl. 435/165; 127/37

(57) ABSTRACT

A method for converting a material comprising lignocellulose or cellulose into a substance convertible with yeast into ethyl alcohol, wherein a mixture comprising 1 part by weight of the material and 0.5 to 5 parts by weight of water is stirred at a temperature of 150 to 270 degrees C. in a vessel closed in terms of pressure in conditions of providing a high shearing force to be pulverized to an average of maximum dimensions of 1 to 20 micrometers, whereby the material is degraded and at least 15% by weight of the cellulose in the material is converted into a substance convertible into ethyl alcohol. It is possible to prepare saccharides for alcohol fermentation from lignocellulose or cellulose by using this method.

### FIELD OF THE INVENTION

The present invention relates to a method for converting a material comprising lignocellulose or cellulose by a mechanical force into a substance convertible into ethyl alcohol. Conversion may hereinafter be referred to as “saccharification”.

### BACKGROUND OF THE INVENTION

Lignocellulose is composed of cellulose bonded to lignin and is difficult to be degraded by cellulase or microorganisms on account of the presence of lignin. Therefore, the lignocelluose is conventionally hydrolyzed with concentrated or diluted sulfuric acid. However, a life time of a plant is half or less due to corrosion by acid in this method. Neutralization is necessary after the hydrolysis, which is costly. Recently, hydrolysis at a subcritical or critical temperature is proposed. Conditions are severe, for instance, a pressure equal to or higher than 22.1 MPa and a temperature equal to or higher than 374 degrees C. Therefore, equipment costs are very high and practical application has not been developed.

Japanese Patent Application Laid-Open No. 2007-104983 describes a pre-processing method for enzymatic hydrolysis of lignocellulose, comprising steps of processing a lignocellulose raw material with an acid; separating solids from liquid; and pulverizing the solids with a beater.

Japanese Patent Application Laid-Open No. Hei-10-327900 describes a method for preparing water-soluble oligosaccharides and monosaccharides from cellulose, comprising steps of bringing cellulose powder into contact with hot pressurized water of 200-300 degrees C. to hydrolyze the cellulose into water-soluble oligosaccharides, which are then enzymatically hydrolyzed.

### PRIOR LITERATURES

Patent Literature 1: Japanese Patent Application Laid-Open No. 2007-104983
Patent Literature 2: Japanese Patent Application Laid-Open No. Hei-10-327900

### SUMMARY OF THE INVENTION

### Problems to be Solved by the Invention

The present invention provides a method for preparing a substance convertible into ethyl alcohol from lignocellulose or cellulose by a mechanical means, wherein no chemical, for instance, acid, is used and an amount of water used is small.

### Means to Solve the Problems

The present invention is a method for converting a material comprising lignocellulose or cellulose into a substance convertible with yeast into ethyl alcohol, wherein a mixture comprising 1 part by weight of the material and 0.5 to 5 parts by weight of water is stirred at a temperature of 150 to 270 degrees C. in a vessel closed in terms of pressure in conditions of providing a high shearing force to be pulverized to an average of maximum dimensions of 1 to 20 micrometers, whereby the material is degraded and at least 15% by weight of the cellulose in the material is converted into a substance convertible into ethyl alcohol.

### Effects of the Invention

According to the present invention, lignocellulose or cellulose is pulverized to 1 to 20 micrometers and, at the same time, cellulose is converted into a substance convertible into ethyl alcohol. Therefore, ethyl alcohol can be prepared in a simple and inexpensive manner.

### BRIEF EXPLANATION OF THE DRAWINGS

FIG. 1 shows a schematic view of the stirring equipment of a closed type which is usable in the present invention.

FIG. 2 shows a graph which shows relation between the torque and the shearing force in the equipment shown in FIG. 1.

FIG. 3 shows a graph showing the output current from the thermocouple over time.

FIG. 4 shows a graph showing the electrical current of the motor over time.

FIG. 5 shows a graph which shows relation between the saccharification degree and the processing temperature, with parameters being a ratio of the calculated amount transmitted forward and the calculated amount transmitted backward in the kneader.

### BEST MODE OF THE INVENTION

The present inventor tried to separate lignin and cellulose by pulverizing a material comprising lignocellulose in a high temperature, high pressure and high shearing force kneader to find that a part of the cellulose converted into a substance convertible into ethyl alcohol with yeast, for instance, a dry yeast.

Further, the inventor has pulverized a material comprising lignocellulose preferably into 100 to 500 micrometers in the high temperature, high pressure and high shearing force kneader, and further processed the material in particular conditions in the kneader, into 1 to 20 micrometers to find that a thermocouple to determine a temperature of the material detected an abnormal electrical current corresponding to a temperature at least 50 degrees C., particularly at least 100 degrees C., more particularly 200 degrees C., higher or lower than a temperature of the material under processing. The inventor analyzed the processed product to find that a considerable portion of the cellulose had been degraded and at least 15% of cellulose had been converted into saccharides convertible into alcohol with yeast. Without being constrained by a particular theory, it is surmised that cellulose is cut by a shearing force to generate radicals which generate electrons which are in turn detected by the thermocouple as the abnormal electrical current. It is surmised that chemical bonds in the cellulose are physically cut by pulverization to 1 to 20 micrometers to generate radicals which cause, in turn, the effective hydrolysis. As a result, lignin dissociates easily from cellulose. The dissociated lignin is separated by extraction. This makes it possible to avoid inhibition of the fermentation by lignin in a subsequent fermentation step with cellulase. At least 20% by weight, preferably at least 35% by weight, more preferably at least 50% by weight, of the cellulose in a filtration residue obtained by filtering the mixture obtained after processing, according to the present invention, the material comprising lignocellulose, has a length which corresponds to 10 to 100 glucose units. This substance having a length which corresponds to 10 to 100 glucose units can be fermented with aspergillus into alcohol and, therefore, fermentation with inexpensive aspergillus is possible instead of expensive cellulase.

The generated radicals further break bonds in the cellulose. The recurring breakage may convert at least 15% by weight, preferably at least 30% by weight, more preferably at least 45% by weight, of the cellulose into a substance convertible with yeast, such as a dry yeast, into ethyl alcohol. A total conversion of the cellulose, including saccharification of the filtration residue which has been degraded into lower molecule with aspergillus, into a substance convertible into ethyl alcohol can be up to at least 20% by weight, preferably up to at least 35% by weight, more preferably up to at least 50% by weight.

Conversion of cellulose into saccharides is seen in the conventional processing of lignocellulose where the lignocellulose is mechanically pulverized in a ball mill as pre-processing for enzymatic hydrolysis and extracted with hot water at a temperature of 230 degrees C. or higher. However, the conversion is at most 15%. Therefore, the results of the present invention are surprising.

In a case of such cellulose-containing material as paper sludge, bean curd lees, sake lees, shochu lees, and agricultural wastes, an effective hydrolysis caused by the pulverization to 1 to 20 micrometers in the processing under heat and by the radicals which are generated by the physical breakage of chemical bonds by the pulverization is enough. In this method the substance convertible into ethyl alcohol with yeast such as a dry yeast alcohol can be prepared from the cellulose-containing material in an inexpensive manner.

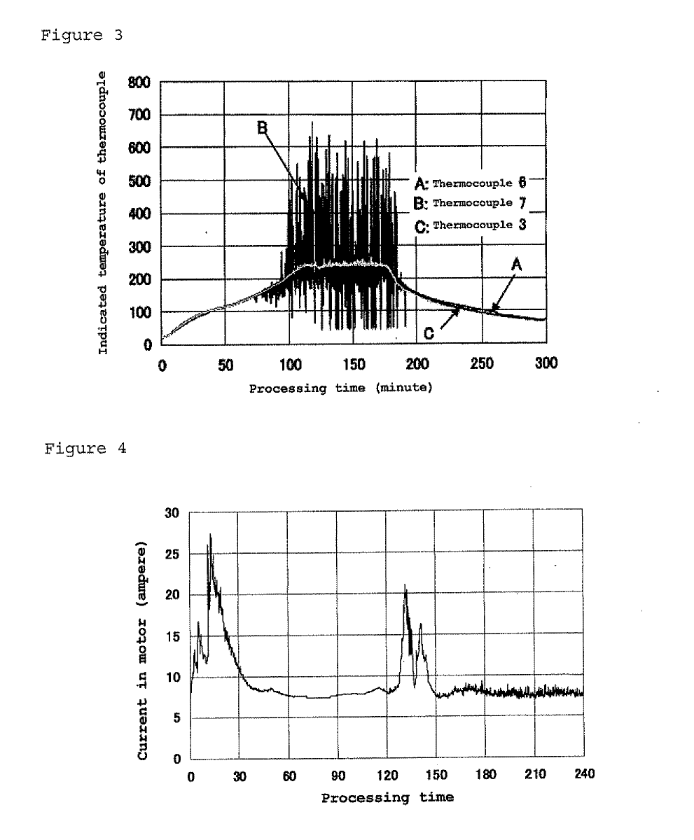

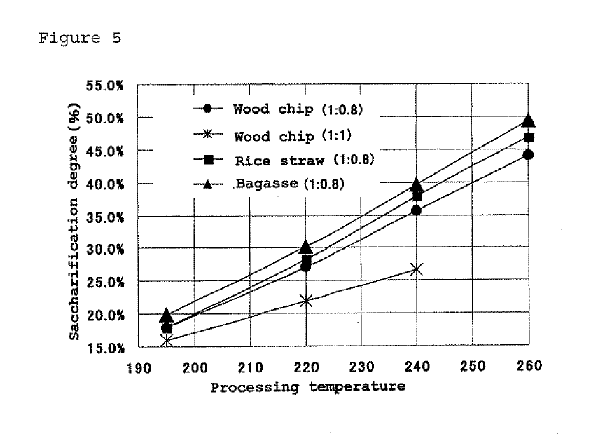

The thermocouple to determine a temperature of the mixture during the stirring detects an abnormal electrical current corresponding to a temperature higher or lower by at least 100 degrees C. than the temperature of the mixture, as shown in FIG. 3. It is surmised that the thermocouple detects the electrons which are released from the radicals generated in the physical breakage of the cellulose bonds with a shearing force. Accordingly, it is concluded that radicals generate and the cellulose is converted into a molecule with a lower molecular weight (see FIG. 5).

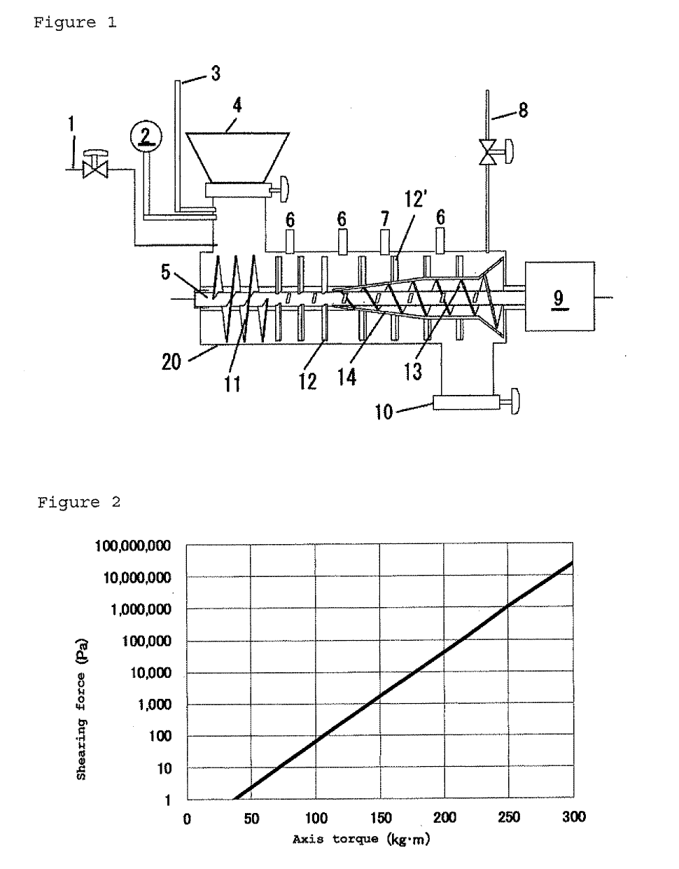

FIG. 1 is a schematic view of an equipment which is suitably used for the present invention. FIG. 1 shows an appearance of the stirring equipment with a cross-sectional view of a cylindrical vessel 20 at the center in its axial direction. Rotating axis 5 which is linked with motor 9 is connected with screw 11 for feeding a raw material through raw material inlet 4, forward blade 12 for transmitting a material to be processed forward in an axis direction, and backward blade 13 for inverting a direction of a flow of the material which has been transmitted forward and transmit it backward in the vicinity of the axis. Each of the forward blades 12 comprise four wings which are attached in an equally spaced manner along the circumference of the axis. Each wing has an angle with the axis so that the material to be processed is transmitted forward when the axis rotates. Forward blade 12′ in the front-side is supported by four elongated attachment boards 14 which are attached to the axis in an equally spaced manner along the circumference of the axis. The material to be processed flows through the spaces between the four elongated attachment boards 14. Backward blade 13 transmits the material to be processed backward when the axis rotates. When a pressure in the vessel exceeds a predetermined value due to steam or other gases which generate in the process, a part of the steam or the gases are released through release pipe 1. The pressure near the inlet is monitored by a bourdon tube pressure gauge. The temperature at the inlet is monitored by thermocouple 3. The temperature and pressure in the processing area in the vessel are monitored by thermocouple and pressure transmitter 6, oil-free pressure sensor, ASG702, of a sanitary type and thermocouple 7. Inert-gas pipe 8 is equipped to feed a high pressure inert gas so that the pressure in the vessel is raised, as will be described below. Further, a jacket, not shown, is equipped so as to heat the vessel.

In an embodiment of the present invention, a mixture comprising a raw material and water is fed gradually from the raw material inlet while rotating axis 5, and transmitted forward by feeding screw 11. When a predetermined amount of the raw material is fed, the raw material inlet is closed. Product outlet 10 is being closed.

In a case where a raw material comprises a material comprising lignocellulose, such as bagasse, rice straws, wheat straws, bamboos, and cores or canes of corns, the initial size of the raw material may be from several millimeters to several hundreds millimeters. This raw material is preferably made smaller beforehand in the aforementioned equipment so that the average of maximum sizes is 100 to 500 micrometers. There, the temperature is preferably 0 to 50 degrees C., more preferably 5 to 30 degrees C., and the pressure is an atmospheric pressure. This step may be referred to as “a pre-pulverization step”.

Then, the step according to the present invention is carried out. In this step, a ratio by weight of raw material to water is 1:0.5-5, preferably 1:1-3. The aforementioned range of the ratio is preferred for effective pre-pulverization.

Then, the vessel is heated so that the temperature of the material to be processed is 150 to 270 degrees C., preferably 160 to 260 degrees C., more preferably 170 to 250 degrees C. In this step according to the present invention, it is important that the raw material is pulverized to the average of maximum dimensions of 1 to 20 micrometers and a shearing force applied to the raw material must be enough for at least 15% by weight of the cellulose in the raw material to be converted to a substance convertible to alcohol with yeast. Such a high shearing force is not attained merely by operating the aforementioned type of equipment without any special ingeniousness. Balance between the ability to transmit the raw material forward by the forward blades and that to transmit it backward by the backward blades is deliberately shifted so that a ratio of the calculated amount of the mixture transmitted forward by the forward blades to the calculated amount of the mixture transmitted backward by the backward blade is 1:0.6 to 0.9, preferably 1:0.65 to 0.85, more preferably 1:0.7 to 0.8. The flow of the raw material is, thereby, disturbed, so that the raw material is vigorously kneaded. Even if the ratio of the amount transmitted forward to the amount transmitted backward is inverse, the same effects may be attained. However, the aforementioned arrangement is easier for design of the equipment. If this ratio is out of the aforementioned range, for instance, 1:1, the high conversion as envisaged in the present invention, that is, at least 15% of the cellulose to a substance convertible into alcohol is not attained. The reason for this is probably that the forward flow and the backward flow in a relatively ordered manner and, therefore, a high shearing force does not generate.

In the present invention, lump lignocellulose and water of an amount of 0.5 to 5 times as that of the lignocellulose are heated to a temperature of 150 to 270 degrees C. at a pressure equal to or higher than a pressure of saturated water vapor at the heating temperature under a shearing force of 0.1 to 20 MPa in a stirring vessel, whereby cellulose in a cellulose-containing material which is relatively softer than lignocellulose, such as paper sludge, bean curd lees, sake lees, shochu lees and agricultural wastes, degrades due to physical decomposition by the shearing and is converted to a material convertible into ethyl alcohol with yeast, as in the case of lignocellulose which is lump and hard.

The present invention also provides a method for easily degrading lignocellulose by cellulose, wherein lignin is made easier to be dissociated from lignocellulose by the method described above and the dissociated lignin is separated by extraction from a filtration residue which contains cellulose which has not much been pulverized (hereinafter referred to as “unpulverized cellulose”).

20% by weight or more, preferably 35% by weight or more, more preferably 50% by weight or more, of the cellulose in the filtration residue obtained by filtering the mixture (slurry) obtained after processing the material comprising lignocellulose has a molecular weight corresponding preferably to 10 to 100 glucose units. Alcohol fermentation thereof by aspergillus can convert at least 15% by weight of the cellulose contained in the degradation residue of cellulose into ethyl alcohol.

An amount of the monosaccharides prepared according to the present method is 1 to 5% of the saccharides generated, as confirmed by high performance liquid chromatography. An amount of xylose is at most 1%. Xylose alone cannot be fermented into alcohol, but xylose as a constituent of oligosaccharides or polysaccharides can be fermented into alcohol.

In the present invention, examples of the material comprising lignocellose include bagasse, timbers from forest thinning, rice straws, wheat straws, bamboos, cores or canes of corns. Examples of the material comprising celluloses include agricultural wastes, paper sludge, bean curd lees, sake lees and shochu lees.

In a preferred embodiment of the present invention, lump lignocellulose of 50 to 200 mm in length and width and 5 to 10 mm in thickness is pre-pulverized to 100 to 500 micrometers and, then, pulverized to 1 to 20 micrometers at a temperature of 150 to 270 degrees C. Even in the step of pre-pulverization, 2 to 5% by weight of the raw material cellulose is saccharified and, therefore, the present pre-pulveriation is preferred more than usual pulverization by, for instance, ball mill, where almost no saccharification occurs. An example of a compact prototype which is suitable for the present invention is, as shown in FIG. 1, a stirring equipment of a closed type having a capacity of 20 litters and provided with a motor of 5.5 kW. In the pre-pulverization, lignocellulose is pulverized to 100 to 500 micrometers at normal temperature and normal pressure. Then, the pulverization according to the present invention is carried out for 5 minutes to 3 hours, preferably 30 minutes to 2 hours, for instance, 60 minutes, so that the lignocellulose is pulverized to 1 to 20 micrometers. In this pulverization step, vapor generates to raise a pressure and, preferably, the pressure is further raised by an inert gas, so that a shearing force is increased and the lignocellulose is pulverized to 1 to 20 micrometers.

The stirring equipment may be any of a batch type and a continuous type which is closed in terms of pressure. The stirring equipment of a continuous type may be any of those which allow continuous feeding of lump lignocellulose, continuous collecting of the generated slurry and continuous release of gases generated such as carbon dioxide and hydrogen, while maintaining the conditions of the present invention.

Water is blended in an amount of 0.5 part by weight or more, preferably 1 part by weight or more, more preferably 1.5 parts by weight or more, and 5 parts by weight or less, preferably 4.5 parts by weight or less, more preferably 4 parts by weight or less, per part by weight of the material comprising lignocellulose or material comprising cellulose. The amount of water to be blended is determined so that the generated slurry is easily taken out and the content of the substance convertible into ethyl alcohol does not exceed 40%.

The upper limit of the heating temperature is 270 degrees C., preferably 260 degrees C., more preferably 250 degrees C. and the lower limit is 150 degrees C., preferably 175 degrees C., more preferably 200 degrees C. If the temperature is higher than the aforementioned upper limit, cellulose causes thermal decomposition and equipment costs are very high. If the temperature is lower than the aforementioned lower limit, effect of the decomposition to the substance capable of alcohol fermentation is not attained. The upper limit of the heating time is preferably 3 hours, more preferably 2 hours, still more preferably 1 hour, particularly preferably 30 minutes. The lower limit is preferably 5 minutes, more preferably 10 minutes, still more preferably 20 minutes. The heating promotes the degradation of lignocellulose or cellulose to a substance capable of alcohol fermentation on account of the hydrolysis by the radicals generated in the physical breakage of chemical bonds which occurs in the pulverization of lignocellulose or cellulose to 1 to 20 micrometers.

The lower limit of the pressure in the pulverization is a pressure higher than a saturated water vapor pressure at the heating temperature, preferably a pressure of the saturated water vapor pressure at the heating temperature+0.1 MPa or higher, more preferably a pressure of the saturated water vapor pressure at the heating temperature+1.0 MPa or higher. The water added is kept in a liquid state at this pressure, so that boiling can be avoided and a shearing force can be maintained. The upper limit of the pressure is a pressure of the saturated water vapor pressure at the heating temperature+3.0 MPa, more preferably a pressure of the saturated water vapor pressure at the heating temperature+2.0 MPa, still more preferably a pressure of the saturated water vapor pressure at the heating temperature+1.5 MPa. The maximum pressure is preferably a pressure of the saturated water vapor pressure at the maximum heating temperature, 270 degrees C., +1.5 MPa, i.e., approximately 7.0 MPa, in order to keep gases, such as carbon dioxide, which generate in the degradation confined. A pressure higher than the upper limit is not preferred because no additional effect is attained and equipment costs increase uselessly. A shearing force increases with increasing pressure. Therefore, a pressure applied on the material is increased in the aforementioned range by, preferably, a degradation gas composed mainly of carbon dioxide which generates from lignocellulose or cellulose by heating, water vapor of the added water, and, more preferably, an inert gas such as nitrogen and argon.

The shearing force in the present invention is raised by pressurizing as described above. The upper limit of the shearing force in the pulverization and pre-pulverization according to the present invention is 20 MPa, preferably 10 MPa, more preferably 5 MPa, still more preferably 3 MPa. The lower limit is 0.1 MPa, preferably 0.3 MPa, more preferably 0.5 MPa. If the shearing force is higher than the aforementioned upper limit, load on the motor is so large that processing costs increase. If the shearing force is lower than the aforementioned lower limit, pre-pulverization is insufficient and cellulose does not degrade sufficiently in the pulverization according to the present invention. The shearing force is provided by the stirring blades equipped in the stirring equipment.

Standard substances with known viscosities at 20 degrees C. for calibrating viscometers according to JIS Z8809 are described in PCT/JP2004/013551, such as JS 100 with a viscosity of 86 mPa·s, JS 14000 with a viscosity of 12 Pa·s and JS 160000 with a viscosity of 140 Pa·s, ex Nippon Grease Co., Ltd. Each of those liquids is placed in the stirring equipment shown in FIG. 1 and the stirring blades equipped are rotated at 20 rpm at 20 degrees C. to determine a torque applied on the rotating axis. For a viscosity of 140 Pa·s at 20 degrees C., mixtures are prepared by blending kerosene with asphalt, for instance, a mixture having a viscosity of 6400 Pa·s at 20 degrees C. as determined on a viscometer of BS type ex Toki Sangyo Co., Ltd., and used for determining the torque, as described above. Here, the aforementioned liquid for the determination is placed in the stirring equipment so that the whole stirring blades are completely immersed in the liquid. A torque is determined also when the stirring equipment is vacant, that is, when no liquid for the determination is placed in the stirring equipment (the shearing force there is supposed to be zero). In this manner, the torque with each of the liquids with known viscosity is read and the shearing force is obtained by the following equation (1):

Shearing force in Pa=[Viscosity in Pa·s multiplied by Shearing speed in s⁻¹]/Torque (1).

Then, the relation between a torque and a shearing force is obtained, as shown, for instance, in FIG. 2. In the aforementioned equation, the shearing speed is expressed by the following equation, wherein the value, sin 3.0 degrees, is specific to the specific stirring equipment shown in FIG. 1. The value is obtained from the shape of the stirring blade and specific to the shape of the stirring blade.

Shearing speed in s⁻¹=1×2×3.14×(rotation number per second)/sin 3.0 degrees (2)

As described above, a shearing force is obtained from the aforementioned relation by determining the torque applied on the rotating axis. An axis torque of a stirring equipment equipped with stirring blades is specific to a particular equipment and, therefore, a different equipment has a different torque. Accordingly, the relation between the torque and the shearing force as shown in FIG. 2 is obtained for each equipment to be used as described above. In this manner, a shearing force is obtained by determining a torque applied on the rotating axis in any equipment.

In the equipment shown in FIG. 1, a flow from the inlet and a flow from the outlet collide with each other to make a flow toward a circumferential wall of the stirring equipment and the intensity of this flow can be detected as a pressure at position 7. It was found that this detected pressure and the shearing force obtained from the determined value of the axis torque in FIG. 2 are same as the values determined using oil-free pressure sensor ASG 702 of a sanitary type, ex Yamatake Corporation.

In the equipment shown in FIG. 1, a lignocellulose material was pulverized to 100 to 500 micrometers in the pre-pulverization using several different stirring blades with a ratio of an amount transmitted backward to an amount transmitted forward of 0.6-1.0:1 and, then, processed at normal temperature and normal pressure for further 1 hour. No change in particle size was observed. Then, pressure was raised up to 1.5 MPa with nitrogen gas at normal temperature and further a shearing processing was carried out for another 1 hour. It was found that the lignocellulose material was pulverized to 1-20 micrometers. Thus, the high pressure increases a shearing force shown.

This phenomenon is shown in FIG. 4. 12 kg of water and 4 kg of wood chips were fed gradually. Around 10 minutes later, a motor current rose close to the maximum, 27 amperes, and 1.0 MPa of a shearing force was applied. With the wood chips being pulverized to 100 to 500 micrometers, the motor current descended. One hour later after the feeding, inlet 4 in FIG. 1 was closed. One hour and 50 minutes later after the feeding, nitrogen gas was introduced over 10 minutes into the stirring equipment through line 8 to a pressure of 1.5 MPa. Immediately after the pressurizing was completed, the motor current began to rise and the shearing force began to appear again, so that the motor current which had been 7.4 amperes increased to 21.5 amperes. At this time, oil-free pressure sensor ASG702 of a sanitary type, ex Yamatake Corporation, showed a shearing force of 1.535 MPa, which is higher by 0.035 MPa than the pressure given by the nitrogen gas. The relation between the axis torque and the shearing force in FIG. 2 shows that a shearing force of 0.035 MPa is applied. Therefore, it is concluded that oil-free pressure sensor ASG702 of a sanitary type, ex Yamatake Corporation, can be used for the determination of a shearing force.

According to the aforementioned present invention, a mixture, i.e., aqueous slurry, is obtained in the stirring equipment after the pulverization, which slurry comprises water which was contained in the lignocellulose; water which had been added in an amount of 0.5 to 5 times the amount of the lignocellulose; unpulverized cellulose; lignin; and polysaccharides including di- or higher saccharides as well as a small amount of monosaccharides. The slurry mixture is filtered to separate a solution comprising saccharides and a filtration residue comprising unpulverized cellulose. This filtration residue is immersed in an organic solvent such as n-hexane and acetone, whereby lignin which was exfoliated or became easier to be exfoliated in a high temperature, high pressure, and high shearing processing according to the present invention can be easily separated. A filtration residue which remains after the separation of lignin is unpulverized cellulose and is easily saccharified with cellulase. In the case of the filtration residue comprising cellulose and lignin which was not separated, at least 20% by weight, preferably at least 35% by weight, more preferably at least 50% by weight, of the filtration residue has been converted to a substance having a molecular weight equivalent to 10 to 100 glucose units. Therefore, at least 20% by weight of the residue can be converted to a substance convertible with aspergillus into ethyl alcohol.

The present invention will be further described in detail, referring to the Examples below, but are not limited thereto.

The lignocelluloses used in Example 1 had the properties described in Table 1 below.

### (Table 1)

### Wood Chip

Size 40 to 50 mm in length and width and 5 to 10 mm in thickness

Water content 13.5% by weight

Rice straws

Size Cut to 50 to 100 mm in length

Water content 3.1% by weight

### Bagasse

Size 10 to 50 mm in length and width and 2 to 4 mm in thickness

Water content 55.6% by weight

The water contents in Table 1 were determined using infrared moisture tester FD-720, ex Kett Electric Laboratory.

For the determination of a shearing force, oil-free pressure sensor ASG702 of a sanitary type, ex Yamatake Corporation, was used.

As a stirring equipment, that shown in FIG. 1 was used. The stirring blades can be changed, whereby a ratio of the calculated amount transmitted forward by the screw feeder at material inlet 4 to the calculated amount transmitted backward from the opposite side is to 1.0:0.6 to 1.0. The equipment had an internal capacity of 20 litters and was equipped with a motor of 5.5 KW. First, while the stirring blades were rotated at 20 rpm, a predetermined amount of the sample and water were fed at normal temperature and normal pressure through material inlet 4. When the feeding was completed, pressure sensor ASG702 (7 in FIG. 1) read 1.0 MPa in gauge pressure (hereinafter, same). Then, while keeping the rotation of the blades at 20 rpm, the equipment was pressurized with nitrogen to 1.8 MPa and, subsequently, heating was started to adjust the processing temperature to 195 degrees C. After the temperature was attained, pressure meter 2 on the side of inlet 4 read 3.0 MPa, which is the saturated water vapor pressure at this temperature, but pressure sensor 7 at the center of the stirring equipment read 3.12 MPa. The motor current was 23 amperes, which is 83.6% of the maximum load. However, as pulverization to a size of 20 micrometers or less proceeded over time and the viscosity of the sample slurry under processing decreased thereby, pressure sensor 7 (for determining a shearing force) indicated the same value as that of pressure meters 2 at inlet 4. The sample was processed for 1 hour, while keeping the temperature in the vessel and the rotation number of the motor at 20 rpm. Then, the content was cooled down to an ambient temperature and the resulting slurry was taken out. The experimental results from RUN-1 to RUN-9 are shown in Table 2.

For comparison in RUN-0, use was made of stirring blades which do not generate a shearing force at all in the process of rising a temperature to 195 degrees C. and in the reaction at 195 degrees C. This proved that the secondary pulverization to 1 to 20 micrometers is essential.

In a case where the secondary pulverization was carried out, the Brix value was 5.5% by weight and the saccharification degree was 27.1% by weight, as shown in Table 2, RUN-1. In a case where no secondary pulverization was carried out in RUN-0, the Brix value after the pre-pulverization increased from 2.1% to 3.3% and the saccharification degree increased only slightly to 14.0% by weight. The pre-pulverization in RUN-0 to RUN-9 was carried out in such conditions that the maximum value of the torque with the 5.5 KW motor was 250 kg.m and the shearing force was 1.0.

In experiments before the present invention was found, the used stirring blades had a ratio of the calculated amount transmitted forward from the material inlet to the calculated amount transmitted backward from the outlet of 1:1. Now in the present invention, the stirring blades had a ratio of the calculated amount transmitted forward to the calculated amount transmitted backward of 1:0.8. In the comparison between the two cases with wood chips shown in FIG. 5, saccharification was increased by about 2% by weight at 195 degrees C. and by about 9% by weight at 240 degrees C.

| | Experiment number | | | | | | | | | | | | |
|---|---|---|---|---|---|---|---|---|---|---|---|---|---|
| | RUN-0 | RUN-01 | RUN-1 | RUN-2 | RUN-3 | RUN-04 | RUN-4 | RUN-5 | RUN-6 | RUN-07 | RUN-7 | RUN-8 | RUN-9 |
| Sample | Wood | Wood | Wood | Wood | Wood | Rice | Rice | Rice | Rice | Bagasse | Bagasse | Bagasse | Bagasse |
| | chips | chips | chips | chips | chips | straws | straws | straws | straws | 195° C. | 220° C. | 240° C. | 260° C. |
| | 220° C. | 195° C. | 220° C. | 240° C. | 260° C. | 195° C. | 220° C. | 240° C. | 260° C. | | | | |
| Processing | No shear- | With a shearing force in the first and secondary pulverizations | | | | | | | | | | | |
| conditions | ing force | | | | | | | | | | | | |
| | in the | | | | | | | | | | | | |
| | secondary | | | | | | | | | | | | |
| | pulver- | | | | | | | | | | | | |
| | ization | | | | | | | | | | | | |
| Processing | 215-225 | 193-198 | 215-225 | 235-247 | 258-264 | 194-198 | 215-225 | 237-248 | 257-266 | 193-199 | 215-225 | 235-248 | 258-270 |
| temperature, C., | | | | | | | | | | | | | |
| at Thermocouple 7 | | | | | | | | | | | | | |
| Processing time | 1 hour | 1 hour | 1 hour | 1 hour | 1 hour | 1 hour | 1 hour | 1 hour | 1 hour | 1 hour | 1 hour | 1 hour | 1 hour |
| Reaction | 3.0 | 3.0 | 3.0 | 4.0 | 5.0 | 3.0 | 3.0 | 4.0 | 5.0 | 3.0 | 3.0 | 4.0 | 5.0 |
| pressure (MPa) | | | | | | | | | | | | | |
| Sample fed | | | | | | | | | | | | | |
| Weight of the | 14.5 | 4.4 | 4.4 | 4.4 | 4.4 | 2.7 | 2.7 | 2.7 | 2.7 | 7.1 | 7.1 | 7.1 | 7.1 |
| sample, kg | | | | | | | | | | | | | |
| Cellulose | 2.7 | 2.7 | 2.7 | 2.7 | 2.7 | 2.0 | 2.0 | 2.0 | 2.0 | 2.3 | 2.3 | 2.3 | 2.3 |
| content in | | | | | | | | | | | | | |
| the sample, kg | | | | | | | | | | | | | |
| Water content in | 76.1 | 13.5% | 13.5% | 13.5% | 13.5% | 3.1% | 3.1% | 3.1% | 3.1% | 55.6% | 55.6% | 55.6% | 55.6% |
| the sample, % | | | | | | | | | | | | | |
| Weight of added | (Saccha- | 12.0 | 12.0 | 12.0 | 12.0 | 9.5 | 9.5 | 9.5 | 10 | 7.9 | 7.9 | 7.9 | 7.9 |
| water, kg | ride con- | | | | | | | | | | | | |
| | centration | | | | | | | | | | | | |
| | 2.1) | | | | | | | | | | | | |
| Total feed | 14.5 | 16.4 | 16.4 | 16.4 | 16.4 | 12.2 | 12.2 | 12.2 | 12.7 | 10.2 | 10.2 | 10.2 | 10.2 |
| amount, kg | | | | | | | | | | | | | |
| Total water | 76% | 76.8% | 76.8% | 75.2% | 76.8% | 78.6% | 78.8% | 78.6% | 79.4% | 90.1% | 90.1% | 90.1% | 90.1% |
| content, wt. % | | | | | | | | | | | | | |
| Slurry | | | | | | — | — | — | | | | | |
| generated | | | | | | | | | | | | | |
| Weight of the | 13.9 | 15.9 | 15.7 | 15.7 | 15.5 | 11.6 | 11.5 | 11.5 | 11.9 | 9.6 | 9.5 | 9.3 | 8.6 |
| generated slurry | | | | | | | | | | | | | |
| which was | | | | | | | | | | | | | |
| recovered, kg | | | | | | | | | | | | | |
| Amount of gas | 0.1 | 0.1 | 0.1 | 0.2 | 0.5 | 0.1 | 0.1 | 0.2 | 0.4 | 0.1 | 0.2 | 0.4 | 1.1 |
| generated, kg | | | | | | | | | | | | | |
| Loss, kg | 0.5 | 0.4 | 0.4 | 0.5 | 0.4 | 0.6 | 0.5 | 0.5 | 0.4 | 0.5 | 0.5 | 0.5 | 0.5 |
| Saccharification | 14.0% | 18.0% | 27.1% | 35.6% | 44.1% | 17.9% | 28.3% | 37.9% | 46.9% | 19.9% | 30.2% | 39.7% | 49.4% |
| degree, wt. % | | | | | | | | | | | | | |
| Brix value, wt. % | 3.3% | 3.7% | 5.5% | 7.3% | 9.1% | 3.5% | 5.5% | 7.4% | 8.8% | 4.7% | 7.1% | 9.4% | 11.6% |
| Water content, | 78.8% | 79.0% | 79.9% | 79.3% | 78.7% | 82.3% | 82.9% | 82.3% | 82.4% | 94.5% | 94.9% | 94.4% | 95.2% |
| wt. % | | | | | | | | | | | | | |

Table 2 shows the properties of the samples, conditions in the processing, and properties and states of the slurries generated. The saccharification degrees in Table 2, that is, (total weight of the filtrate from a squeezing device×saccharide concentration in %)/(weight of cellulose in the sample fed), were calculated from the saccharide concentrations of the filtrates obtained by filtering the slurries generated. The saccharide concentrations were determined at normal temperature, using saccharide concentration meter PAL-1, ex Atago Co., Ltd. Here, the cellulose content in the sample was obtained from the weight of lignin which was extracted with n-hexane from the residue of the slurry generated and the water content of the starting material. The weight of gases generated was obtained from a volume of the gas and composition analysis by gas chromatography after cooling down the reaction equipment, which contained a slurry comprising a pressurizing nitrogen gas or a degradation gas, to normal temperature.

In order to confirm whether the value calculated from the value indicated by the saccharide meter corresponds to the weight of the obtained saccharides and whether the saccharides which can be used for alcohol fermentation were generated, 50 ml of the slurry generated from wood chips at 220 degrees C. in RUN-1, 50 ml of the slurry generated from rice straws at 220 degrees C. in RUN-4, and 50 ml of a 6% solution of sucrose as a reference were each placed in Erlenmeyer flasks. Contents of saccharide in the first two slurries were 5.5%. 1 g of yeast, ex Oriental Yeast Co., Ltd., was added in each flask and water was added up to a whole volume of 100 ml in order to reduce the viscosities of the slurries. The flasks were plugged with rubber stoppers equipped with a Tedlar (trademark) bag for 1 litter so that an amount of carbon dioxide generated can be measured. Fermentation process was carried out at 40 degrees C. for 72 hours in a compact shaking incubator PIC-100, ex AS ONE Co., Ltd. In the measurement of carbon dioxide generated, it was confirmed that the generated carbon dioxide gas corresponded to approximately a half, precisely 48.5 to 49.1%, of the saccharides generated. Then, this fermentation liquid was filtered and the filtrate was subjected to measurement with a concentration meter of a boiling point type, BMS-L850-12, ex Takara Thermistor Co., Ltd. The alcohol concentration by volume percentage was 1.68 for the slurry generated at 220 degrees C. from wood chips in RUN-1; 1.64 for the slurry generated at 220 degrees C. from rice straws in RUN-4; and 1.84 for sucrose (conversion to alcohol: 49.1%). From these results, it was confirmed that the values measured by a pocket saccharide meter, PAL-1, ex Atago Co., Ltd., were significant.

It was possible to obtain alcohol whose amount corresponds to 9 to 13.9% of a remainder of taking the weight of water contained in the starting material fed from the weight of the saccharified liquid which had been subjected to the shearing process at 240 to 260 degrees C.

It was confirmed that the slurry generated from wood chips, rice straws, and bagasse at 240 degrees C. and 260 degrees C. contained inhibitors against alcohol fermentation, such as acetaldehyde, hydroxymethylfrufral and vanillin. The saccharified product was put in a reduced pressure by a rotary evaporator and, then, subjected to fermentation. It was confirmed that these substances were easily removed by a rotary evaporator. Therefore, it was confirmed that these inhibitors were easily removed by depressurizing and taking out the resulting slurry at 110 to 150 degrees C. after the high shearing process at 240 to 270 degrees C.

It was confirmed that a small amount of the inhibitors which were left behind after taking out in the aforementioned temperature range could be removed by adding 1 to 3% by weight of charcoal relative to the slurry generated.

Lignin dissociated from cellulose by heat and a shearing force. It was confirmed that the cellulose could be isolated from the filtration residue comprising lignin and cellulose by extracting lignin with n-hexane.

In order to investigate whether starch or saccharides which were left in dried koji rice, wherein koji rice means steamed rice with aspergillus propagating thereon, was fermented into alcohol, 200 g of water, 10 g of dried koji rice, 1 g of a dry yeast and 2 g of yogurt for preventing saprogen from propagating were placed in a bottle and left at room temperature, about 25 to 30 degrees C., for 10 days while stirring three times a day. It was confirmed that 80.2%, 8.02 g by weight, of the starch or saccharides which was probably contained in the dried koji rice turned into alcohol. Then, the slurry obtained in RUN-8 was filtered and the residue was dried. 100 g of the dried residue, 200 g of water, 10 g of dried koji rice (trade name: Miyakokoji, ex Isesou Co., Ltd., rice with aspergillus dried at a low temperature), 1 g of a dry yeast and 2 g of yogurt for preventing saprogen from propagating were placed in a bottle and left at room temperature, about 25 to 30 degrees C., while stirring three times a day. On day 2, it began to smell of unrefined sake. On day 4, it began to smell of alcohol, the sample which had been viscous began to be soft and a transparent liquid emerged on the surface. On day 10, the content was filtrated with compression and an alcohol content in the filtrate was determined to be 13.4 by a concentration meter of a boiling point type, BMS-L850-12, ex Takara Thermista Co., Ltd. It was found that about 42 g of the starting material residue and about 8 g of the starch or saccharides contained in the dried koji rice was fermented to alcohol, 24.5 g.

### Example 2

As a starting material, 5 kg of toilet paper comprising about 100% of cellulose was used. This toilet paper was immersed in 10 kg of water and placed in the experimental equipment. The equipment used in the experiment had the same stirring blades as those used in Example 1, where a ratio of the calculated amount transmitted forward to the calculated amount transmitted backward was 1:0.8. Experimental conditions were 220 degrees C. and 1 hour. The experimental results are shown in Table 3. A saccharification rate was 30.4%. A yield of alcohol based on the starting material toilet paper was about 15% because the saccharification rate was 30.4%. As described above, saccharification of cellulose which does not comprise lignin by a high shearing force kneader is easier than that of lignocellulose.

| Experiment number | RUN-10 | |
|---|---|---|
| Experiment sample | Toilet paper | |
| | 220° | C. |
| Processing conditions | | |
| Processing temperature, C. | 215-225 | |
| Processing time | 1 | hour |

| | |
|---|---|
| Reaction pressure, MPa | 3.0 |
| Sample fed | |
| Weight of the sample, kg | 5.0 |
| Cellulose content in the sample, kg | 4.8 |
| Water content in the sample, % | 3.1% |
| Weight of water added, kg | 12.0 |
| Total feed amount, kg | 17.0 |
| Total water content, wt. % | 71.5% |
| Slurry generated | |
| Weight of the slurry generated | 16.5 |
| which was recovered, kg | |
| Amount of gas generated, kg | 0.1 |
| Loss, kg | 0.4 |
| Saccharification degree, wt. % | 30.4% |
| Brix value, wt. % | 11.7% |
| Water content, wt. % | 73.3% |

### INDUSTRIAL APPLICABILITY

According to the present invention, a mixture comprising a lump lignocellulose material with a length and width of 50 to 200 mm and a thickness of 5 to 10 mm, such as bagasse, timbers from forest thinning, rice straws, wheat straws, bamboos, and cores or canes of corns, and water in an amount of 0.5 to 5 times the amount of the material is pulverized with a high shearing force. Increase in pressure with increasing temperature in the heating process or further pressurization with an inert gas such as nitrogen and argon further increase a shearing force to cause pulverization to 20 micrometers or less. It has been found that this pulverization physically breaks chemical bonds to generate radicals which cause effective degradation. Further, by radicals which are generated in the pulverization of paper sludge, bean curd lees, sake lees, shochu lees or agricultural wastes with a lower shearing force, saccharides are effectively obtained by degradation which takes place in parallel with the dissociation of lignin from cellulose and the radical degradation of the cellulose. Further, the present invention provides a method for obtaining cellulose, wherein lignin which has become easier to be dissociated from the unsaccharified cellulose in the filtration residue is isolated by extraction with n-hexane.

The amount of the monosaccharides generated is 1 to 5% of the resulting saccharides. The amount of xylose generated is at most 1%. Xylose alone cannot be fermented into alcohol, but xylose as a constituent of oligosaccharides or polysaccharides can be fermented into alcohol in the present method.

Further, in the case of the residue comprising cellulose associated with lignin, a half of the filtration residue is converted into lower molecules having a molecular weight equivalent to arabinose composed of approximately 100 glucoses. Therefore, the present invention is a method for preparing ethyl alcohol, wherein the filrtraion residue is fermented by aspergillus, whereby at least 15% by weight of the degradation residue of the cellulose can be fermented into alcohol.

Where a mixture after saccharification of a material comprising lignocellulose is stirred at a temperature higher than 200 degrees C. and a thermocouple to determine a temperature of the mixture detects an abnormal electric current corresponding to a temperature which is higher or lower by at least 300 degrees C. than a temperature of the mixture, a small amount of inhibitors against alcohol fermentation, such as acetaldehyde, hydroxymethylfurfural and vanillin generate. Almost all of these inhibitors can be removed together with generated gases such as carbon dioxide by depressurizing the mixture at 100 to 105 degrees C. A small amount of the inhibitors left behind can be removed by adding 1 to 3%, based on the slurry generated, of charcoal to the slurry generated.

Saccharides for alcohol fermentation can be prepared from lignocellulose or cellulose in this method.

### DESCRIPTION OF THE SYMBOLS IN THE DRAWINGS

1. Outlet for water vapor/generated gas
2. Bourdon pipe pressure gauge
3. Thermocouple
4. Material inlet
5. Stirring wings capable of providing a shearing force
6. Thermocouple and pressure transmitter
7. Oil free-pressure sensor ASG702 of a sanitary type and thermocouple
8. Pressurizing line by an inert gas
9. Motor
10. Material outlet
11. Feeding blade
12. Forward blade
13. Backward blade
14. Attachment board

1. A method for converting a material comprising lignocellulose or cellulose into a substance convertible with yeast into ethyl alcohol, wherein a mixture comprising 1 part by weight of the material and 0.5 to 5 parts by weight of water is stirred at a temperature of 150 to 270 degrees C. in a vessel closed in terms of pressure in conditions of providing a high shearing force to be pulverized to an average of maximum dimensions of 1 to 20
micrometers, whereby the material is degraded and at least 15% by weight of the cellulose in the material is converted into a substance convertible into ethyl alcohol.

2. The method according to claim 1, wherein the vessel is cylindrical and comprises a rotating axis extending in a direction of a center axis, and forward blades and backward blades equipped on the rotating axis and wherein a ratio of a calculated amount of the mixture transmitted forward by the forward blades to calculated amount of the mixture transmitted backward by the backward blades is 1:0.6 to 0.9.

3. The method according to claim 2, wherein the aforementioned ratio is 1:0.65 to 0.85.

4. The method according to claim 3, wherein the aforementioned ratio is 1:0.7 to 0.8.

5. The method according to claim 1, wherein the shearing force of 0.1 to 20 MPa is applied to the material by stirring.

6. The method according to claim 5, wherein the shearing force is 0.3 to 10 MPa.

7. The method according to claim 1, wherein a thermocouple to measure a temperature of the mixture during the stirring detects an abnormal electric current corresponding to a temperature at least 50 degrees C. higher or lower than a temperature of the material.

8. The method according to claim 7, wherein the thermocouple to measure the temperature of the mixture during the stirring detects an abnormal electric current corresponding to a temperature at least 100 degrees C. higher or lower than a temperature of the material.

9. The method according to claim 1, wherein a raw material has an average of maximum dimensions of 100 to 500 micrometers.

10. The method according to claim 1, wherein a pressure in the vessel is made higher than a saturated water vapor pressure at the temperature, using a pressurized inert gas.

11. The method according to claim 1, wherein the pressure in the vessel in the pulverization is higher than the saturated water vapor pressure by 0.1 to 3.5 MPa, provided that the pressure in the vessel is equal to or less than a gauge pressure of 7.0 MPa.

12. The method according to claim 1, wherein the pulverization step is carried out for 5 minutes to 3 hours.

13. The method according to claim 1, wherein the material comprising lignocellulose is selected from the group consisting of bagasse, timbers from forest thinning, rice straws, wheat straws, bamboos, cores or canes of corns and the material comprising cellulose is selected from the group consisting of agricultural wastes, paper sludge, bean curd lees, sake lees and shochu lees.

14. The method according to claim 1, wherein 20% by weight or more of the cellulose in filtration residue comprising cellulose obtained after filtrating the mixture obtained after the processing of the material comprising lignocellulose has a molecular weight corresponding to 10 to 100 glucose units.

15. A method for preparing ethyl alcohol, wherein the mixture obtained by the method according to claim 1 is subjected to alcohol fermentation with yeast.

16. A method for preparing ethyl alcohol, wherein the mixture obtained according to claim 1 is filtered and the filtrate is subjected to alcohol fermentation with yeast.

17. The method for preparing ethyl alcohol according to claim 16, wherein the filtration residue is subjected to saccharification by aspergillus and the resulting saccharide is subjected to alcohol fermentation with yeast.

18. The method according to claim 1, wherein the mixture is subjected to stripping at a temperature of 110 to 150 degrees C. at a normal pressure to remove at least a part of a material inhibiting alcohol fermentation with yeast and, then, subjected to alcohol fermentation with yeast.

19. The method according to claim 18, wherein 1 to 3% by weight, based on the mixture for fermentation after the stripping, of charcoal powder is added to the mixture and then the mixture is subjected to alcohol fermentation.
